(19) United States
(12) Patent Application Publication
(10) Pub. No.: US 2020/0008713 A1
(43) Pub. Date: Jan. 9, 2020

(54) WALKING SUPPORT SYSTEM, WALKING SUPPORT METHOD, AND PROGRAM

(71) Applicant: HONDA MOTOR CO., LTD., MINATO-KU, TOKYO (JP)

(72) Inventor: Toru TAKENAKA, WAKO-SHI, SAITAMA (JP)

(21) Appl. No.: 16/491,025

(22) PCT Filed: Mar. 12, 2018
(86) PCT No.: PCT/JP2018/009475

§ 371 Date: Sep. 4, 2019

(30) Foreign Application Priority Data
Mar. 15, 2017 (JP) 2017-050148

(51) Int. Cl. A61B 5/11; A61B 5/00; G06F 3/01

(52) U.S. Cl. CPC A61B 5/112; G06F 3/017; G06F 3/011; A61B 5/742

(57) ABSTRACT

A walking support system for supporting a user while walking includes a display unit, a landing timing detection unit configured to detect a timing of a landing while the user walks and a display control unit configured to cause the display unit to display an auxiliary image for prompting the user to change an angle of his/her upper body in synchronization with the timing of the landing on the basis of an output of the landing timing detection unit.

### TECHNICAL FIELD

The present invention relates to a walking support system, a walking support method, and a program.

Priority is claimed on Japanese Patent Application No. 2017-050148, filed Mar. 15, 2017, the content of which is incorporated herein by reference.

### BACKGROUND ART

Conventionally, a system in which a human body is equipped with a sensor or the like to measure a walking state of the person has been considered. For example, Patent Literature 1 discloses a walking state system for measuring a variation of the center of gravity and a variation of a joint angle of a leg associated with walking, calculating an index indicating a walking motion of a user on the basis of the measured variation of the center of gravity, the measured variation of the joint angle, and the user's body information that does not change due to the walking, and displaying the calculated index.

### CITATION LIST

### Patent Literature

### [Patent Literature 1]

Japanese Unexamined Patent Application, First Publication No. 2012-65723

### SUMMARY OF INVENTION

### Technical Problem

However, in the technology described in Patent Literature 1, technology for enabling a user to walk smoothly on the basis of a measured walking state of the user is not considered. Also, in the case of the aged and handicapped, the inability to accelerate the upper body at an appropriate timing while walking may hinder smooth walking.

An aspect according to the present invention has been made in view of the above-described circumstances and an objective of the present invention is to provide a walking support system, a walking support method, and a program for promoting an appropriate motion of the upper body of a user while walking and guiding the user for a smooth walking motion.

### Solution to Problem

In order to solve the above-described technical problem and achieve the objective, the present invention adopts the following aspects.

(1) According to an aspect of the present invention, there is provided a walking support system for supporting a user while walking, the walking support system including: a display unit; a landing timing detection unit configured to detect a timing of a landing while the user walks; and a display control unit configured to cause the display unit to display an auxiliary image for prompting the user to change an angle of his/her upper body in synchronization with the timing of the landing on the basis of an output of the landing timing detection unit.

(2) In the above-described aspect (1), the display control unit may be configured to cause the display unit to display the auxiliary image for prompting the user to change the angle of his/her upper body in a direction of raising his/her upper body at the timing of the landing.

(3) In the above-described aspect (1) or (2), the display control unit may be configured to cause the display unit to display the auxiliary image for prompting the user to change the angle of his/her upper body in a direction of bending his/her upper body forward at a middle timing between landings.

(4) In the above-described aspect (2), the display control unit may be configured to cause the display unit to display the auxiliary image in which a change is made so that an object disposed in front of the user's field of view is close to the user at the timing of the landing.

(5) In the above-described aspect (2), the display control unit may be configured to cause the display unit to display the auxiliary image in which a virtual grid disposed within the user's field of view or an object on the virtual grid moves onto the virtual grid above a current position.

(6) In the above-described aspect (2), the display control unit may be configured to cause the display unit to display the auxiliary image in which part of the user's field of view is shielded above the user's field of view.

(7) In the above-described aspect (2), the display control unit may be configured to cause the display unit to display a prescribed object of interest as the auxiliary image above the user's field of view.

(8) In the above-described aspect (1), the display control unit may be configured to cause the display unit to display an object on a side of the user's field of view and to display the auxiliary image in which the object rotates around an axial line extending in a horizontal direction.

(9) In any one of the above-described aspects (1) to (8), the walking support system may be configured to further include an upper body angle detection unit configured to detect the angle of the user's upper body while the user walks, and the display control unit may be configured to cause the display unit to display the auxiliary image for prompting the user to change the angle of his/her upper body on the basis of an output of the upper body angle detection unit.

(10) In any one of the above-described aspects (1) to (9), the walking support system may be configured to further include an upper body angle detection unit configured to detect the angle of his/her upper body while the user walks and the display control unit may be configured to cause an image showing the angle of the user's upper body at a prescribed timing and an image showing a standard angle of his/her upper body at the prescribed timing to be displayed on the basis of an output of the landing timing detection unit and an output of the upper body angle detection unit.

(11) According to an aspect of the present invention, there is provided a walking support method including: detecting, by a control computer of a walking support system, a timing of a landing while a user walks; and causing, by the control computer of the walking support system, a display unit to display an auxiliary image for prompting the user to change an angle of his/her upper body in synchronization with the timing of the landing.

(12) According to an aspect of the present invention, there is provided a program for causing a control computer of a walking support system to execute: a process of detecting a landing while a user walks; and a process of displaying an auxiliary image for prompting the user to change an angle of his/her upper body in synchronization with a timing of the landing.

### Advantageous Effects of Invention

According to an aspect of the present invention, it is possible to promote an appropriate motion of the upper body of a user while walking and guide the user for a smooth walking motion.

### BRIEF DESCRIPTION OF DRAWINGS

FIG. 1 is a diagram showing an outline of a walking support system.

FIG. 2 is a diagram showing an outline of a walking support system according to a first embodiment.

FIG. 3 is a block diagram showing an example of a configuration of the walking support system according to the first embodiment.

FIG. 4 is a first flowchart showing an example of a process of the walking support system according to the first embodiment.

FIG. 5 is a second flowchart showing an example of a process of the walking support system according to the first embodiment.

FIG. 6 is a first diagram showing an example of an auxiliary image according to the first embodiment.

FIG. 7 is a second diagram showing an example of an auxiliary image according to the first embodiment.

FIG. 8 is a third diagram showing an example of an auxiliary image according to the first embodiment.

FIG. 9 is a fourth diagram illustrating an example of an auxiliary image according to the first embodiment.

FIG. 10 is a fifth diagram illustrating an example of an auxiliary image according to the first embodiment.

FIG. 11 is a first flowchart illustrating an example of a process of a walking support system according to a second embodiment.

FIG. 12 is a second flowchart showing an example of a process of the walking support system according to the second embodiment.

FIG. 13 is a diagram showing an example of a display image according to a third embodiment.

### DESCRIPTION OF EMBODIMENTS

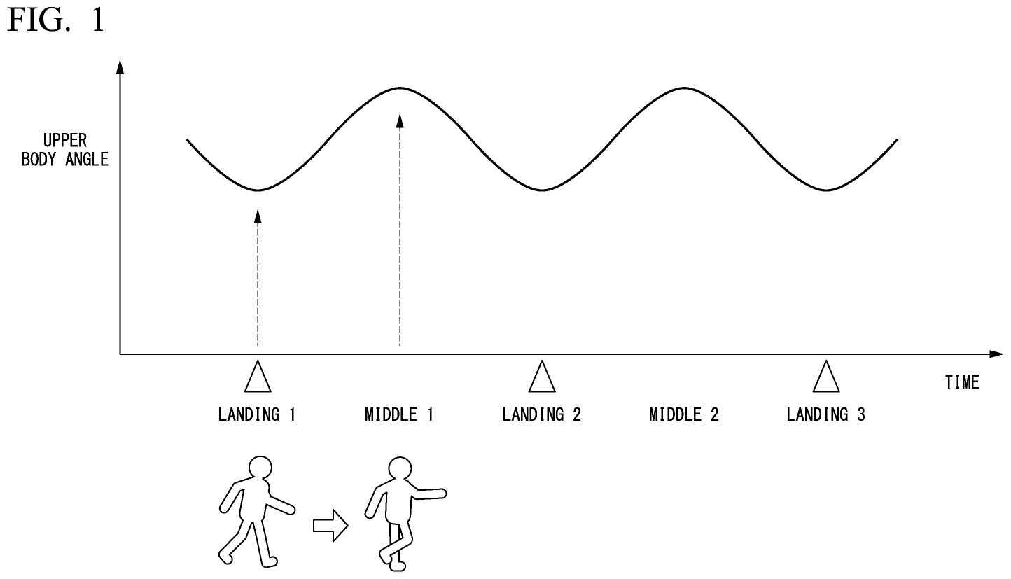

First, an outline of the present embodiment will be described. FIG. 1 is a diagram showing an outline of a walking support system. FIG. 1 shows an example of a relationship between a landing timing of a pedestrian and an upper body angle. The landing timing is a timing when one foot of the pedestrian is in contact with the ground (makes a landing). The upper body angle is an angle of the upper body (an upper half of the body) of the pedestrian with respect to the ground. In FIG. 1, the horizontal axis represents time and a right direction indicates the elapse of time. The vertical axis represents the upper body angle of the user and represents an increasing angle of his/her upper body, i.e., the upper body of the pedestrian becoming closer to a position perpendicular to the ground, as his/her upper body goes upward. Also, points indicated by triangles indicate individual landing timings of the pedestrian.

While walking, ideally, the upper body angle becomes a minimum (inclined furthest forward) at the landing or immediately after the landing and the upper body angle becomes a maximum (closet to a perpendicular angle) at a middle timing between landings or immediately after the middle timing. In the example of FIG. 1, the upper body angle at landing 1 becomes a minimum, the upper body angle at middle 1 becomes a maximum, and the upper body angle also becomes a minimum at landing 2. Thus, while walking, smooth walking is implemented by appropriately linking the landing and the upper body angle.

However, for example, elderly people with weak muscle strength, people with leg paralysis, and the like may not be able to walk smoothly because the landing and the upper body angle are not appropriately linked. For example, when the upper body is inclined forward more greatly after the landing, the entire center of gravity cannot be moved sufficiently forward, the stride becomes smaller, and smooth walking cannot be consequently performed. Also, the field of view may be narrowed because the forward inclination of the upper body is not returned from.

In order to solve the above-described problems, the walking support system according to the present embodiment acquires a landing timing of the user and displays an auxiliary image for prompting the user to change the angle of his/her upper body on the basis of the acquired landing timing of the user in synchronization with the landing timing of the user. More specifically, the walking support system displays an auxiliary image for prompting the user to make a change in a direction of raising his/her upper body in accordance with the landing timing of the user. Thereby, the user is prompted to raise his/her upper body quickly after the landing and can implement smooth walking.

### First Embodiment

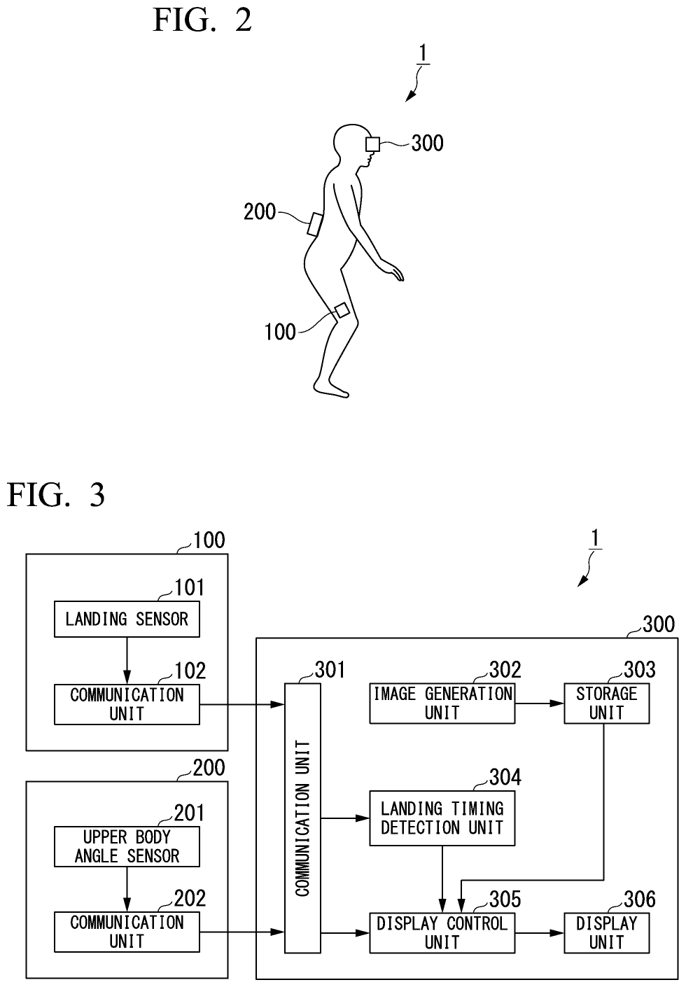

Next, the configuration of the first embodiment will be described. FIG. 2 is a diagram showing an outline of a walking support system 1 according to the first embodiment of the present invention. The walking support system 1 includes a landing detection device 100, an upper body angle detection device 200, and a display device 300.

The landing detection device 100 includes, for example, an acceleration sensor. The landing detection device 100 is worn on a leg or the like of the user and acquires information for detecting a landing timing of the user. The landing detection device 100 may be worn on a foot or shoe of the user.

The upper body angle detection device 200 includes, for example, an inclination sensor including an angular speed sensor and an acceleration sensor. The upper body angle detection device 200 is worn on a waist, a back, or the like of the user in parallel to a width direction of the user's body and acquires information for detecting the angle of the user's upper body.

The display device 300 is an augmented reality (AR) device configured to display additional information in a reality space visually recognized by the user. Also, the display device 300 may also be a virtual reality (VR) device configured to display virtual reality. The display device 300 is, for example, a glasses-type display or a head-mounted display worn on the head of the user. The display device 300 displays an auxiliary image for prompting the user to change the angle of his/her upper body on the basis of the information acquired from the landing detection device 100 or the upper body angle detection device 200.

The landing detection device 100 and the upper body angle detection device 200 are connected to the display device 300 so that communication can be performed in a wired or wireless manner. Also, the landing detection device 100, the upper body angle detection device 200, and the display device 300 may be configured as the same device. Also, the landing detection device 100, the upper body angle detection device 200, and the display device 300 may be configured as some of functions of a smartphone or the like.

FIG. 3 is a block diagram of the walking support system 1 according to the present embodiment. The landing detection device 100 includes a landing sensor 101 and a communication unit 102.

The landing sensor 101 acquires information for detecting a landing timing of the user. The landing sensor 101 is, for example, an acceleration sensor, and detects acceleration acting on the landing sensor 101. Because the landing detection device 100 is worn on the user's leg, the acquired acceleration represents the acceleration of the user's leg. The landing sensor 101 outputs the acquired acceleration to the communication unit 102. Also, the landing sensor 101 is a sensor such as an angular speed sensor, a geomagnetic sensor, or a vibration sensor, and may acquire information other than acceleration and output the information to the communication unit 102.

The communication unit 102 includes a communication interface for performing communication between devices via a wired or wireless network and communicates with the communication unit 301 of the display device 300. The communication unit 102 outputs the acceleration of the user's leg input from the landing sensor 101 to the communication unit 301.

The upper body angle detection device 200 includes an upper body angle sensor 201 and a communication unit 202. The upper body angle sensor 201 detects an angle of the user's upper body with respect to the ground. The upper body angle sensor 201 is, for example, a combination of an angular speed sensor, an acceleration sensor, and an integral computing unit, calculates the angle of the user's upper body by performing an integral arithmetic process on a detected angular speed, and further corrects the calculated angle of the upper body using the acceleration sensor. Also, the upper body angle sensor 201 may detect the angle of the user's upper body with respect to the user's lower body on the basis of acquired information of an angle sensor attached to the user's hip joint or the like. The upper body angle sensor 201 outputs the acquired angle of the user's upper body to the communication unit 202.

The communication unit 202 includes a communication interface for performing communication between devices via a wired or wireless network and communicates with the communication unit 301 of the display device 300. The communication unit 202 outputs the angle of the user's upper body input from the upper body angle sensor 201 to the communication unit 301.

The display device 300 includes a communication unit 301, an image generation unit 302, a storage unit 303, a landing timing detection unit 304, a display control unit 305, and a display unit 306. The image generation unit 302, the landing timing detection unit 304, and the display control unit 305 are implemented, for example, by a processor such as a central processing unit (CPU) executing a program. Also, some or all of these components are implemented, for example, by hardware such as large scale integration (LSI), an application specific integrated circuit (ASIC), or a field-programmable gate array (FPGA), or may be implemented by cooperation between software and hardware.

The communication unit 301 includes a communication interface for performing communication between devices via a wired or wireless network and communicates with the communication unit 102 of the landing detection device 100 and the communication unit 202 of the upper body angle detection device 200. The communication unit 301 outputs the acceleration of the user's leg input from the communication unit 102 to the landing timing detection unit 304. Also, the communication unit 301 outputs the angle of the user's upper body input from the communication unit 202 to the display control unit 305.

The image generation unit 302 generates an auxiliary image for prompting the user to change the angle of his/her upper body. The auxiliary image is additionally displayed on the reality space visually recognized by the user. Also, the auxiliary image may be additionally displayed within the virtual space displayed by the display device 300. Further, the auxiliary image may be a still image of one frame or a moving image (a video) including a plurality of frames. A specific example of the auxiliary image will be described below. The image generation unit 302 outputs the generated auxiliary image to the storage unit 303. Although the image generation unit 302 outputs the auxiliary image created in advance to the storage unit 303 asynchronously with the landing timing of the user, the auxiliary image may be generated in synchronization with the landing timing of the user.

The storage unit 303 includes, for example, a hard disc drive (HDD), a flash memory, an electrically erasable programmable read only memory (EEPROM), a read only memory (ROM), a random access memory (RAM), and the like. The storage unit 303 stores various types of programs to be executed by a processor such as a CPU provided in the display device 300 such as firmware and an application program, a result of processing executed by the processor, and the like. The storage unit 303 holds the auxiliary image input from the image generation unit 302 and outputs the auxiliary image to the display control unit 305 in response to a request from the display control unit 305. Also, the storage unit 303 may output an auxiliary image pre-registered from the outside to the display control unit 305.

The landing timing detection unit 304 acquires the acceleration of the user's leg input from the landing detection device 100 via the communication unit 301. The landing timing detection unit 304 detects the landing timing of the user on the basis of the acquired acceleration. The landing timing detection unit 304, for example, calculates a speed of the user's leg by performing an integral arithmetic process on the acquired acceleration, and detects a timing at which a downward speed changes from positive to negative as the landing timing of the user. Alternatively, a timing at which the acceleration suddenly changes to a prescribed value or more is detected as the landing timing of the user. The landing timing detection unit 304 outputs the detected landing timing of the user to the display control unit 305. A process of the landing timing detection unit 304 may be performed by the landing detection device 100 and the landing timing of the user detected by the landing detection device 100 may be acquired and output to the display control unit 305. Also, the landing timing detection unit 304 may detect the landing timing using a means for estimating a phase of walking, for example, the technology described in Japanese Patent No. 5938124.

The display control unit 305 controls a function related to image display of the display device 300. Specifically, the display control unit 305 controls the display unit 306 so that various types of images including the auxiliary image are displayed. Details of an operation of the display control unit 305 will be described below.

The display unit 306 is, for example, a glasses-type display or a head-mounted display, and displays various types of images including an auxiliary image on the display on the basis of control of the display control unit 305. The display unit 306 may two-dimensionally display the auxiliary image on a transmissive display or may three-dimensionally displays the auxiliary image using a 3D display of a polarization glasses type, a liquid crystal shutter glasses type, or the like. Also, the display unit 306 may display the auxiliary image on an external screen by projection without using a display, or may display a stereoscopic image using optical technology such as holography. In this case, the display unit 306 is not required to be worn on the user.

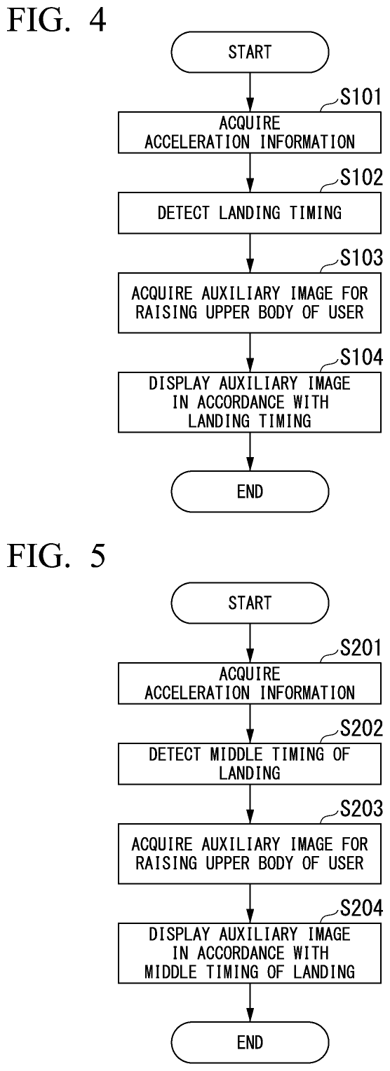

Next, an operation of the walking support system 1 according to the present embodiment will be described. FIG. 4 is a first flowchart showing an example of a process of the walking support system 1 according to the present embodiment.

First, the landing timing detection unit 304 of the display device 300 acquires acceleration of the user's leg input from the landing detection device 100 via the communication unit 301 (step S101).

Next, the landing timing detection unit 304 detects a landing timing of the user on the basis of the acquired acceleration (step S102). Thereafter, the landing timing detection unit 304 outputs the detected landing timing of the user to the display control unit 305.

When the landing timing of the user is input from the landing timing detection unit 304, the display control unit 305 acquires an auxiliary image for raising the user's upper body from the storage unit 303 (step S103). The display control unit 305 may pre-acquire the auxiliary image from the storage unit 303 and hold the auxiliary image.

Next, the display control unit 305 causes the display unit 306 to display the auxiliary image for raising the user's upper body in accordance with the acquired landing timing of the user (step S104). An example of the display of the auxiliary image will be described below. Also, the display control unit 305 may cause the auxiliary image to be displayed at each landing timing or may cause the auxiliary image to be displayed in accordance with the predicted landing timing by predicting the next landing timing on the basis of the landing timing acquired during a prescribed period. This is the end of the description of FIG. 4.

Subsequently, another operation of the walking support system 1 according to the present embodiment will be described. FIG. 5 is a second flowchart showing an example of a process of the walking support system 1 according to the present embodiment.

First, the landing timing detection unit 304 of the display device 300 acquires acceleration of the user's leg input from the landing detection device 100 via the communication unit 301 (step S201).

Next, the landing timing detection unit 304 detects a middle timing between landing timings of the user on the basis of the acquired acceleration (step S202). The middle timing between landings can be obtained by adding half of a walking cycle of an immediately previous step to an immediately previous landing timing. At this time, an average of walking cycles up to several steps ago may be used instead of the walking cycle of the immediately previous step. Alternatively, a timing at which the user's upper body passes through the foot of the support leg may be detected as the middle timing between the landings. Thereafter, the landing timing detection unit 304 outputs the detected middle timing of the landing of the user to the display control unit 305.

When the middle timing of the landing of the user is input from the landing timing detection unit 304, the display control unit 305 acquires an auxiliary image for bending the user's upper body forward from the storage unit 303 (step S203).

Next, the display control unit 305 causes the display unit 306 to display the auxiliary image for bending the user's upper body forward in accordance with the acquired middle timing of the landing of the user (step S204). An example of the display of the auxiliary image will be described below. This is the end of the description of FIG. 5.

Also, the walking support system 1 may perform the process of FIG. 4 and the process of FIG. 5 in combination.

That is, the walking support system 1 may display the auxiliary image for raising the user's upper body in accordance with the landing timing of the user and display the auxiliary image for bending the user's upper body forward in accordance with the middle timing between the landing timings of the user.

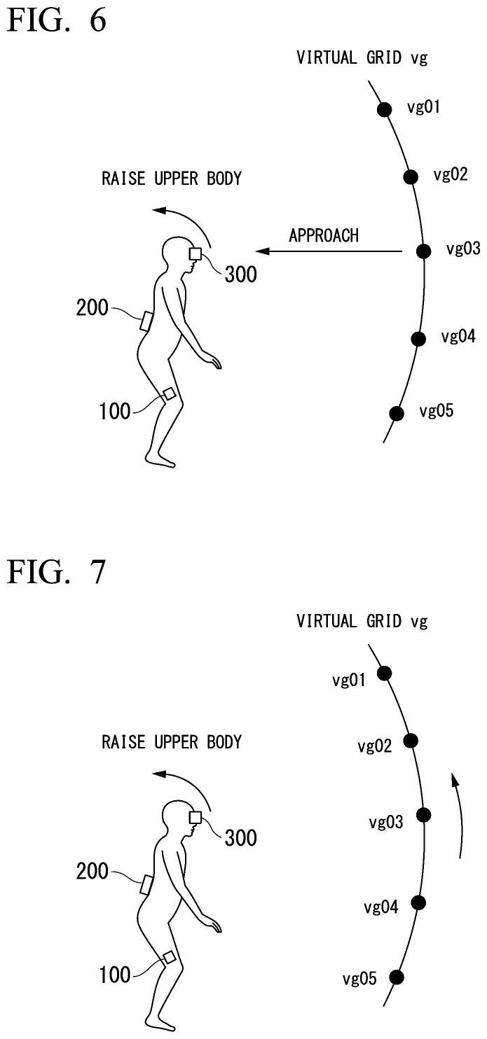

Next, an auxiliary image according to the present embodiment will be described. FIG. 6 is a first diagram showing an example of the auxiliary image according to the present embodiment. Points vg01 to vg05 in FIG. 6 are generated by the image generation unit 302 of the display device 300 and represent intersections of a grid-like virtual grid vg virtually displayed in front of the user's field of view. The virtual grid vg is, for example, virtually disposed so that the virtual grid vg exists on a spherical surface surrounding the user. Also, the virtual grid vg may be virtually disposed on a vertical plane in front of the user.

In the example of FIG. 6, the display control unit 305 causes a video in which the virtual grid vg approaches in the user direction to be displayed as an auxiliary image in accordance with the landing timing of the user. Thereby, a case in which the user has an illusion that his/her head is moving forward with respect to the virtual grid vg and moves his/her head backward to eliminate the illusion is conceived. As a result, it is possible to prompt the user to raise his/her upper body. Therefore, the user can raise his/her upper body quickly after the landing and can walk smoothly.

Also, the display control unit 305 may cause a video in which the virtual grid vg is moved away from the user to be displayed as the auxiliary image in accordance with the middle timing of the landing of the user. Thereby, a case in which the user has an illusion that his/her head is moving backward with respect to the virtual grid vg and moves his/her head forward to eliminate the illusion is conceived. As a result, it is possible to prompt the user to bend his/her upper body forward. Therefore, the user can bend his/her upper body quickly at the middle timing of the landing and can walk smoothly. Also, the display control unit 305 may promote appropriate acceleration of the user's upper body by causing another virtual object instead of the virtual grid vg to be close to or away from the user.

FIG. 7 is a second diagram showing an example of the auxiliary image according to the present embodiment. As in FIG. 6, the display device 300 displays a virtual grid vg in front of the user's field of view. In the example of FIG. 7, the display control unit 305 causes an image in which the virtual grid vg slides upward along a disposed spherical surface to be displayed as an auxiliary image in accordance with the landing timing of the user. When the virtual grid vg is disposed on a vertical plane in front of the user, the display control unit 305 causes a video sliding upward along the vertical plane on which the virtual grid vg is disposed to be displayed. Thereby, a case in which the user has an illusion that his/her head is lowered with respect to the virtual grid vg and moves his/her head upward to eliminate the illusion is conceived. As a result, it is possible to prompt the user to raise his/her upper body. Therefore, the user can raise his/her upper body quickly after the landing and can walk smoothly.

The display control unit 305 may cause a video which slides downward along a spherical surface or a vertical surface on which the virtual grid vg is disposed to be displayed as an auxiliary image in accordance with a middle timing of the landing of the user. Thereby, a case in which the user has an illusion that his/her head is raised with respect to the virtual grid vg and moves his/her head downward to eliminate the illusion is conceived. As a result, it is possible to prompt the user to bend his/her upper body forward. Therefore, the user can quickly bend his/her upper body forward at a middle timing of the landing and can walk smoothly.

Also, the display control unit 305 may promote appropriate acceleration of the user's upper body by rotating the virtual grid vg upward or downward along the spherical surface on which the virtual grid vg is disposed and displaying the virtual grid vg. Also, the display control unit 305 may also promote appropriate acceleration of the user's upper body by disposing another virtual object on the virtual grid vg and moving the virtual object upward or downward along the virtual grid.

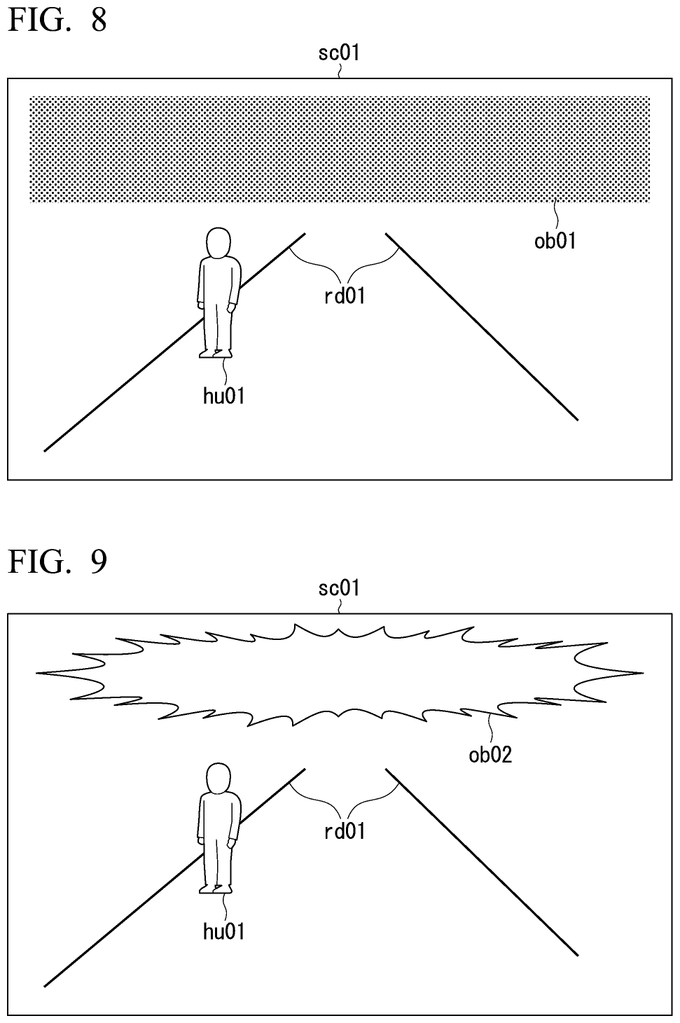

FIG. 8 is a third diagram showing an example of an auxiliary image according to the present embodiment. In FIG. 8, sc01 shows an image of the display unit 306 superimposed on the user's field of view. Also, a human hu01 and a road rd01 may be an actual human and road displayed via the display device 300 or may be those virtually displayed by the display device 300.

The display control unit 305 causes a shielding object ob01 for shielding (masking) part of the user's field of view in an upper portion within the display screen sc01 to be displayed as an auxiliary image in accordance with the landing timing of the user. The shielding object ob01 is, for example, a grid-like or mesh-like image, and shields the part of the user's field of view. Also, the shielding object ob01 is a translucent image, a blinking image, an image subjected to mosaic processing, or the like, and also includes an image that lowers forward visibility of the user. Because the obscuration object ob01 lowers the forward visibility, a case in which the user reflectively tries to gaze at the front of the shielding object ob01 displayed above the field of view and moves his/her head upward is conceived. As a result, it is possible to prompt the user to raise his/her upper body. Therefore, the user can raise his/her upper body quickly after the landing and can walk smoothly.

The display control unit 305 may cause the shielding object ob01 to be displayed in a lower portion within the display screen sc01 as an auxiliary image in accordance with a middle timing of the landing of the user. Thereby, a case in which the user reflectively tries to gaze at the front of the shielding object ob01 displayed below the field of view and moves his/her head downward is conceived. As a result, it is possible to prompt the user to bend his/her upper body forward. Therefore, the user can quickly bend his/her upper body forward at a middle timing of the landing and can walk smoothly.

FIG. 9 is a fourth diagram showing an example of an auxiliary image according to the present embodiment. As in FIG. 8, the display device 300 displays various types of images within a display screen sc01. The display control unit 305 causes an object ob02 of interest for attracting the user's attention to be displayed as an auxiliary image in an upper portion within the display screen sc01 in accordance with a timing of a landing of the user. The object ob02 of interest is, for example, an image for attracting the user's visual attention such as a character image, a colored image, or a prescribed mark or sign. Within the object ob02 of interest, a keyword for attracting the user's attention may be displayed. Also, a specific instruction such as “please raise upper body” may be displayed within the object ob02 of interest. Thereby, a case in which the user reflectively tries to gaze at the object ob02 of interest displayed above the field of view and moves his/her head upward is conceived. As a result, it is possible to prompt the user to raise his/her upper body. Therefore, the user can raise his/her upper body quickly after the landing and can walk smoothly.

The display control unit 305 may cause the object ob02 of interest to be displayed as an auxiliary image in a lower portion within the display screen sc01 in accordance with a middle timing of the landing of the user. Thereby, a case in which the user reflectively tries to gaze at the object ob02 of interest disposed below the field of view and moves his/her head downward is conceived. As a result, it is possible to prompt the user to bend his/her upper body forward. Therefore, the user can quickly bend his/her upper body forward at a middle timing of the landing and can walk smoothly.

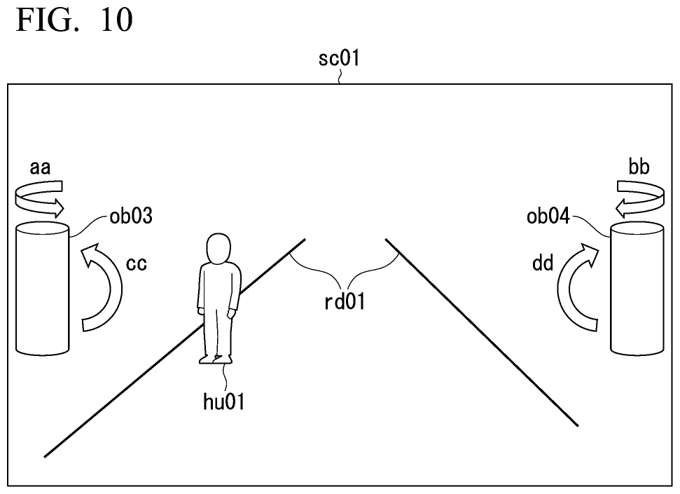

FIG. 10 is a fifth diagram illustrating an example of an auxiliary image according to the present embodiment. The display control unit 305 causes an object ob03 and an object ob04 to be displayed on the left and right within the display screen sc01. The display control unit 305 may cause the object ob03 and the object ob04 to be displayed on the sides of the display screen sc01 all the time or may cause the object ob03 and the object ob04 to be displayed only near the landing timing of the user.

The display control unit 305 causes the object ob03 and the object ob04 to rotate in a direction opposite to a direction in front of the user, i.e., a traveling direction, in accordance with the landing timing of the user. In other words, the display control unit 305 causes the object ob03 and the object ob04 to rotate around an axial line extending in a horizontal direction. In the example of FIG. 9, the display control unit 305 causes the object ob03 to rotate in a direction of an arrow aa and causes the object ob04 to rotate in a direction of an arrow bb. Thereby, the user is prompted to reflectively pull his/her head backward in accordance with the rotation of the object ob02 and the object ob03 in accordance with the landing timing and the user is consequently prompted to raise his/her upper body. Therefore, the user can raise his/her upper body quickly after the landing and can walk smoothly.

The display control unit 305 may cause the object ob03 to rotate in a counterclockwise direction (a direction of an arrow cc) and cause the object ob04 to rotate in a clockwise direction (a direction of an arrow dd), in accordance with the landing timing of the user. Thereby, the user is prompted to raise the gaze upward in accordance with the rotation of the object ob02 and the object ob03 in a reflective manner in accordance with the landing timing and the user is consequently prompted to raise his/her upper body. Therefore, the user can raise his/her upper body quickly after the landing and can walk smoothly.

The display control unit 305 may cause the object ob03 and the object ob04 to rotate in a direction opposite to those of the above-described arrows aa and bb in accordance with the middle timing of the landing of the user. Also, the display control unit 305 may cause the object ob03 and the object ob04 to rotate in a direction opposite to those of the above-described arrows cc and dd in accordance with the middle timing of the landing of the user. Thereby, the user is prompted to reflectively bend his/her upper body forward in accordance with the rotation of the object and the user can quickly bend his/her upper body forward at a middle timing of the landing and can walk smoothly.

As described above, according to the present embodiment, there is provided the walking support system 1 for supporting a user while walking, the walking support system including: the display unit 306; the landing timing detection unit 304 configured to detect a timing of a landing while the user walks; and the display control unit 305 configured to cause the display unit 306 to display an auxiliary image for prompting the user to change an angle of his/her upper body in synchronization with the timing of the landing on the basis of an output of the landing timing detection unit 304. Thereby, it is possible to promote an appropriate motion of the user's upper body while walking and guide the user for a smooth walking motion.

Also, in the walking support system 1 according to the present embodiment, the display control unit 305 causes the display unit 306 to display the auxiliary image for prompting the user to change the angle of his/her upper body in a direction of raising his/her upper body at the timing of the landing. Thereby, it is possible to prompt the user to raise his/her upper body quickly after the landing and guide the user for a smooth walking motion.

Also, in the walking support system 1 according to the present embodiment, the display control unit 305 causes the display unit 306 to display the auxiliary image for prompting the user to change the angle of his/her upper body in a direction of bending his/her upper body forward at a middle timing between landings. Thereby, it is possible to prompt the user to bend the upper body forward at the middle timing between the landings and guide the user for a smooth walking motion.

Also, in the walking support system 1 according to the present embodiment, the display control unit 305 causes the display unit 306 to display the auxiliary image in which a change is made so that an object disposed in front of the user's field of view is close to the user at the timing of the landing. Thereby, it is possible to prompt the user to raise his/her upper body quickly after the landing and guide the user for a smooth walking motion.

Also, in the walking support system 1 according to the present embodiment, the display control unit 305 causes the display unit 306 to display the auxiliary image in which a virtual grid disposed within the user's field of view or an object on the virtual grid moves onto the virtual grid above a current position. Thereby, it is possible to prompt the user to raise his/her upper body quickly after the landing and guide the user for a smooth walking motion.

Also, in the walking support system 1 according to the present embodiment, the display control unit 305 causes the display unit 306 to display the auxiliary image in which part of the user's field of view is shielded above the user's field of view. Thereby, it is possible to prompt the user to raise his/her upper body quickly after the landing and guide the user for a smooth walking motion.

Also, in the walking support system 1 according to the present embodiment, the display control unit 305 causes the display unit 306 to display a prescribed object of interest as the auxiliary image above the user's field of view. Thereby, it is possible to prompt the user to raise his/her upper body quickly after the landing and guide the user for a smooth walking motion.

Also, in the walking support system 1 according to the present embodiment, the display control unit 305 causes the display unit 306 to display an object on a side of the user's field of view and to display the auxiliary image in which the object rotates around an axial line extending in a horizontal direction. Thereby, it is possible to promote an appropriate motion of the user's upper body while walking and guide the user for a smooth walking motion.

### Second Embodiment

Hereinafter, a second embodiment of the present invention will be described with reference to the drawings. Also, components similar to those of the above-described embodiment are denoted by the same reference signs and description thereof is adopted here. A configuration of a walking support system 2 according to the present embodiment is similar to that of the walking support system 1 according to the first embodiment. In addition to the process in the first embodiment, the walking support system 2 determines the display of an auxiliary image using an angle of an upper body of a user.

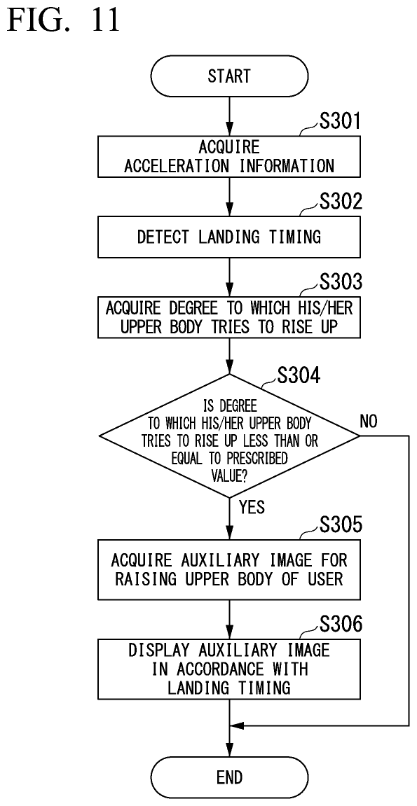

FIG. 11 is a first flowchart showing an example of a process of the walking support system 2 according to the present embodiment.

First, a landing timing detection unit 304 of a display device 300 acquires acceleration of a leg of a user input from a landing detection device 100 via a communication unit 301 (step S301).

Next, the landing timing detection unit 304 detects a landing timing of the user on the basis of the acquired acceleration (step S302). Thereafter, the landing timing detection unit 304 outputs the detected landing timing of the user to a display control unit 305.

Next, the display control unit 305 acquires a degree to which the user tries to raise his/her upper body (hereinafter may be abbreviated as a degree to which his/her upper body tries to rise up) input from the upper body angle detection device 200 via the communication unit 301 (step S303). The degree to which the user tries to raise his/her upper body is represented by a low-cut value for an inclination of his/her upper body, an angular speed of the inclination of his/her upper body, or an angular speed of the inclination of his/her upper body. Alternatively, the degree is represented by a linear combination value thereof.

Next, the display control unit 305 compares the acquired degree to which the user's upper body tries to rise up with a prescribed value (step S304). When the degree to which the user's upper body tries to rise up is less than or equal to the prescribed value, the process proceeds to the processing of step S305. When the degree to which the user's upper body tries to rise up is greater than the prescribed value, the process ends.

When the degree to which the user's upper body tries to rise up is less than or equal to the prescribed value, the display control unit 305 acquires an auxiliary image for raising the user's upper body from the storage unit 303 (step S305). Also, the display control unit 305 may pre-acquire the auxiliary image from the storage unit 303 and hold the auxiliary image.

Next, the display control unit 305 causes the display unit 306 to display the auxiliary image for raising the user's upper body in accordance with the acquired landing timing of the user (step S306). Thereafter, the process ends.

In the process of FIG. 11, the walking support system 2 acquires an upper body angle of the user in addition to the landing timing of the user and determines whether or not to display the auxiliary image for raising the user's upper body. Accordingly, for example, when the user's upper body is sufficiently raised even immediately after the landing, the walking support system 2 does not display the auxiliary image for raising the user's upper body. Thus, the walking support system 2 according to the present embodiment can more appropriately display the auxiliary image for raising the user's upper body. Also, an auxiliary image for raising the user's upper body more strongly may be displayed when the degree to which his/her upper body tries to rise up is lower. Thereby, the user's upper body can be raised more appropriately.

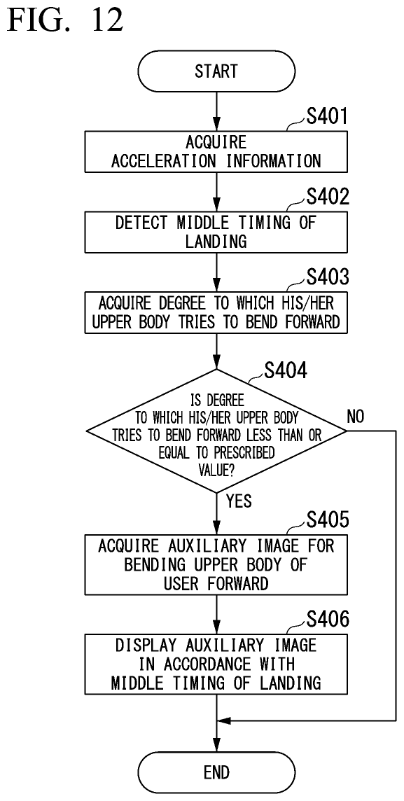

Subsequently, another operation of the walking support system 2 according to the present embodiment will be described. FIG. 12 is a second flowchart showing an example of a process of the walking support system 2 according to the present embodiment.

First, the landing timing detection unit 304 of the display device 300 acquires the acceleration of the user's leg input from the landing detection device 100 via the communication unit 301 (step S401).

Next, the landing timing detection unit 304 detects a middle timing of the landing of the user on the basis of the acquired acceleration (step S402). Thereafter, the landing timing detection unit 304 outputs the detected middle timing of the landing of the user to the display control unit 305.

Next, the display control unit 305 determines the degree to which the user tries to bend his/her upper body forward (hereinafter may be abbreviated as a degree to which his/her upper body tries to be bent forward) input from the upper body angle detection device 200 via the communication unit 301 (step S403). It is only necessary for the degree to which the user tries to bend his/her upper body forward to be a value obtained by multiplying the degree to which the user tries to raise his/her upper body by (−1).

Next, the display control unit 305 compares the acquired degree to which the user's upper body tries to be bent forward with a prescribed value (step S404). When the degree to which the user's upper body tries to be bent forward is less than or equal to the prescribed value, the process proceeds to the processing of step S305. When the degree to which the user's upper body tries to be bent forward is greater than the prescribed value, the process ends.

The display control unit 305 acquires an auxiliary image for bending the user's upper body forward from the storage unit 303 when the degree to which his/her upper body tries to be bent forward is greater than or equal to the prescribed value (step S405). Also, the display control unit 305 may pre-acquire the auxiliary image from the storage unit 303 and hold the auxiliary image.

Next, the display control unit 305 causes the display unit 306 to display the auxiliary image for bending the user's upper body forward in accordance with the acquired middle timing of the landing of the user (step S406). Thereafter, the process ends.

In the process of FIG. 12, the walking support system 2 acquires an upper body angle of the user in addition to the middle timing of the landing of the user and determines whether or not to display an auxiliary image for bending the user's upper body forward. Accordingly, the walking support system 2 does not cause an auxiliary image for bending the user's upper body forward to be displayed, for example, when the user's upper body is sufficiently inclined forward even at a timing between landings. Thus, the walking support system 2 according to the present embodiment can cause the auxiliary image for bending the user's upper body forward to be more appropriately displayed. Also, an auxiliary image for bending the user's upper body forward more strongly may be displayed when the degree to which his/her upper body tries to be bent forward is lower. Thereby, the user's upper body can be bent forward more appropriately.

As described above, the walking support system 2 according to the present embodiment includes the upper body angle detection unit (the upper body angle detecting device 200) configured to detect the angle of the user's upper body while the user walks in addition to the function of the walking support system 1. The display control unit 305 causes the display unit 306 to display an auxiliary image for prompting the user to change the angle of his/her upper body on the basis of the output of the upper body angle detection unit (the upper body angle detection device 200). Thereby, it is possible to promote an appropriate motion of the user's upper body while walking and guide the user for a smooth walking motion in consideration of the upper body angle of the user.

### Third Embodiment

Hereinafter, a third embodiment of the present invention will be described with reference to the drawings. Also, components similar to those of the above-described embodiment are denoted by the same reference signs and description thereof is adopted here. A configuration of a walking support system 3 according to the present embodiment is similar to that of the walking support system 1 according to the first embodiment. In addition to the process in the first embodiment, the walking support system 3 displays an image showing an angle of an upper body of a user at a prescribed timing and an image showing a standard angle of his/her upper body at the timing.

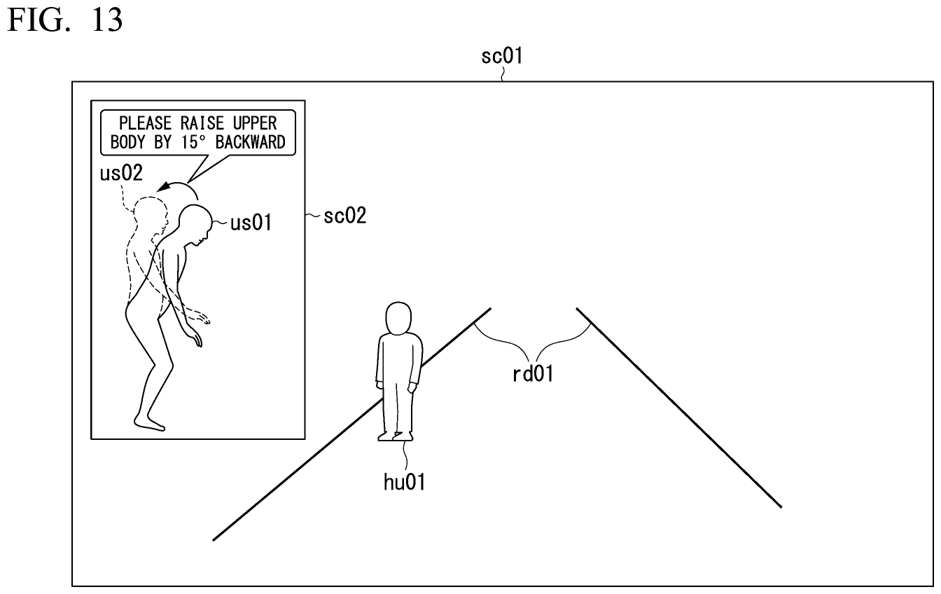

FIG. 13 is a view showing an example of a display image according to the present embodiment. In the example of FIG. 12, a display control unit 305 of a display device 300 causes a sub-display-screen sc02 to be displayed on the left side within a display screen sc01. Within the sub-display-screen sc02, a user image us01 obtained by viewing the user from the side and a reference image us02 are displayed.

An image generation unit 302 generates the user image us01 on the basis of a current angle of the user's upper body acquired from an upper body angle detection device 200 and the display control unit 305 causes the display unit 306 to display the user image us01. That is, the user image us01 is an image representing the current angle of the user's upper body. The image generation unit 302 generates the reference image us02 on the basis of an ideal angle of the upper body corresponding to a current walking motion (landing timing) of the user acquired from a landing sensor 101 and the display control unit 305 causes the display unit 306 to display the reference image us02. Also, the ideal angle of the upper body corresponding to the current walking motion (landing timing) of the user is pre-stored in the storage unit 303. That is, the reference image us02 is an image representing an angle of the upper body serving as a current standard (target) of the user.

The user image us01 and the reference image us02 may be still images at a specific timing or may be videos that change in real time in accordance with the walking motion of the user. Also, the display control unit 305 may hide one of the user image us01 and the reference image us02. Also, the user image us01 and the reference image us02 may be displayed not only in real time while walking but also on demand after the end of walking.

As described above, in addition to the function of the walking support system 1, the walking support system 3 according to the present embodiment further includes an upper body angle detection unit (the upper body angle detection device 200) configured to detect an angle of the upper body while the user walks. The display control unit 305 causes an image showing the angle of the user's upper body at a prescribed timing and an image showing a standard angle of his/her upper body at the timing to be displayed on the basis of an output of the landing sensor 101 and an output of an upper body angle sensor 201. Thereby, the user can objectively ascertain the angle of his/her upper body while walking and the standard angle of his/her upper body and can implement a smoother walking motion in which the angle of his/her upper body is close to an ideal angle.

Although the embodiments of the present invention have been described above in detail with reference to the drawings, the specific configurations are not limited to the embodiments and design changes and the like are also included without departing from the scope of the present invention. For example, the order of processing procedures, sequences, flowcharts, and the like in the respective embodiments may be changed as long as there is no inconsistency.

Also, an aspect of the present invention can be variously modified within the scope of the claims and an embodiment obtained by appropriately combining the technical means respectively disclosed in different embodiments is also included in a technical scope of the present invention. Also, a configuration in which an element is replaced between elements described in the above-described embodiments and modified examples and exhibiting similar effects is also included therein.

Also, the above-described embodiment may be used in combination with a walking assist device. The walking assist device is a walking training device configured to support efficient walking on the basis of an “inverted pendulum model”. In the walking assist device, a motion of a hip joint of the user while walking is detected by angle sensors built in left and right motors and a control computer drives the motors. Thereby, guidance for the swing-out of a lower limb of the user by bending of his/her hip joint and guidance for the kick-out of the lower limb of the user by extension are performed. By using the present embodiment in combination with the walking assist device, it is possible to appropriately guide the user for the motion of his/her upper body that cannot be covered by the walking assist device and to perform walking assistance more effectively.

### REFERENCE SIGNS LIST

1, 2, 3 Walking support system
100 Landing detection device
101 Landing sensor
102, 202, 301 Communication unit
200 Upper body angle detection device
201 Upper body angle sensor
300 Display device
302 Image generation unit
303 Storage unit
304 Landing timing detection unit
305 Display control unit
306 Display unit

1. A walking support system for supporting a user while walking, the walking support system comprising:
a display unit;
a landing timing detection unit configured to detect a timing of a landing while the user walks; and
a display control unit configured to cause the display unit to display an auxiliary image for prompting the user to change an angle of his/her upper body in synchronization with the timing of the landing on the basis of an output of the landing timing detection unit.

2. The walking support system according to claim 1, wherein the display control unit causes the display unit to display the auxiliary image for prompting the user to change the angle of his/her upper body in a direction of raising his/her upper body at the timing of the landing.

3. The walking support system according to claim 1, wherein the display control unit causes the display unit to display the auxiliary image for prompting the user to change the angle of his/her upper body in a direction of bending his/her upper body forward at a middle timing between landings.

4. The walking support system according to claim 2, wherein the display control unit causes the display unit to display the auxiliary image in which a change is made so that an object disposed in front of the user's field of view is close to the user at the timing of the landing.

5. The walking support system according to claim 2, wherein the display control unit causes the display unit to display the auxiliary image in which a virtual grid disposed within the user's field of view or an object on the virtual grid moves onto the virtual grid above a current position.

6. The walking support system according to claim 2, wherein the display control unit causes the display unit to display the auxiliary image in which part of the user's field of view is shielded above the user's field of view.

7. The walking support system according to claim 2, wherein the display control unit causes the display unit to display a prescribed object of interest as the auxiliary image above the user's field of view.

8. The walking support system according to claim 1, wherein the display control unit causes the display unit to display an object on a side of the user's field of view and to display the auxiliary image in which the object rotates around an axial line extending in a horizontal direction.

9. The walking support system according to claim 1, further comprising an upper body angle detection unit configured to detect the angle of the user's upper body while the user walks,
wherein the display control unit causes the display unit to display the auxiliary image for prompting the user to change the angle of his/her upper body on the basis of an output of the upper body angle detection unit.

10. The walking support system according to claim 1, further comprising an upper body angle detection unit configured to detect the angle of his/her upper body while the user walks,
wherein the display control unit causes an image showing the angle of the user's upper body at a prescribed timing and an image showing a standard angle of his/her upper body at the prescribed timing to be displayed on the basis of an output of the landing timing detection unit and an output of the upper body angle detection unit.

11. A walking support method comprising:
detecting, by a control computer of a walking support system, a timing of a landing while a user walks; and
causing, by the control computer of the walking support system, a display unit to display an auxiliary image for prompting the user to change an angle of his/her upper body in synchronization with the timing of the landing.

12. A program for causing a control computer of a walking support system to execute:
a process of detecting a landing while a user walks; and
a process of displaying an auxiliary image for prompting the user to change an angle of his/her upper body in synchronization with a timing of the landing.
